(19) United States
(12) Patent Application Publication
(10) Pub. No.: US 2023/0092846 A1
(43) Pub. Date: Mar. 23, 2023

(54) COMBINATION FOR THE TREATMENT OF CANCER AND APPLICATION THEREOF

(71) Applicant: EVERFRONT BIOTECH INC., New Taipei City (TW)

(72) Inventors: Tzyy-Wen CHIOU, City New Taipei City (TW); Horng-Jyh HARN, New Taipei City (TW); Shinn-Zong LIN, New Taipei City (TW); Jui-Hao Lee, New Taipei City (TW); Jen-Wei Liu, New Taipei City (TW); Szu-Yin Lin, New Taipei City (TW)

(73) Assignee: EVERFRONT BIOTECH INC., New Taipei City (TW)

(21) Appl. No.: 17/792,468

(22) PCT Filed: Jan. 14, 2021
(86) PCT No.: PCT/CN2021/071685

§ 371 Date: Sep. 7, 2022

Related U.S. Application Data
(60) Provisional application No. 62/961,131, filed on Jan. 14, 2020.

(51) Int. Cl. A61K 31/365; A61P 35/00

(52) U.S. Cl. CPC A61K 31/365; A61P 35/00

(57) ABSTRACT

A combination, comprising a first component and a second component; the first component is selected from a group composed of the following: the compound of formula (I), a pharmaceutically acceptable salt thereof, and a combination thereof, wherein A is a C1-C8 aliphatic hydrocarbon group optionally containing a carbonyl group as needed; X is H or OH; Y is O; and R₁ is H or is not present, the condition being that when R₁ is not present, Y and A bond to form a five-membered ring; and the second component is selected from a group composed of the following: a topoisomerase inhibitor, a microtubule assembly inhibitor, a platinum-based agent, an antimetabolite, and a combination thereof.

### FIELD

The present invention relates to cancer treatment. More specifically, the present invention relates to uses of a compound of formula (I) and/or a pharmaceutically acceptable salt of the compound of formula (I) in combination with an anticancer medicament for cancer treatment, especially for enhancing a sensitivity of cancer cells to the anticancer medicament, reducing a side effect of the anticancer medicament, reversing an immunosuppression induced by the anticancer medicament and/or attenuating a symptom of cachexia in a subject with cancer:

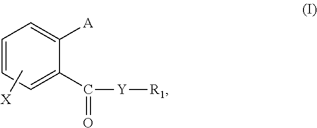

wherein A is a C1-C8 aliphatic hydrocarbyl optionally having a carbonyl; X is H or OH; Y is O; and R₁ is H or absent, with the proviso that when R₁ is absent, Y and A bond together to form a five-membered ring.

### BACKGROUND

In medicine, a tumor refers to an abnormal lesion of cells. Genetically, under the action of various carcinogenic factors, the cells of a local tissue in the body cannot normally regulate their growth, thereby causing the cells abnormally grow and accumulate to a lump, which is called as “tumor.” Cancer is also called as malignant tumor. In addition to accumulating into a lump, the abnormally grown cancer cells will further diffuse and metastasize to other tissues or organs in the body. Since the growth and metastasis of cancer cells will cause serious physiological dysfunction which is hard to be cured completely, cancer has become the top cause of global human death in recent years.

Regarding the treatment of cancer, the common clinical therapies in current comprise surgery, chemotherapy, radiotherapy, target therapy, immunotherapy, etc., wherein a chemical medicament, such as a topoisomerase inhibitor, a microtubule assembly inhibitor, a platinum-based agent, and/or an antimetabolite is used in the chemotherapy to kill the fast-growing cancer cells. However, most of the medicaments used in chemotherapy also affect normal cells and influence their growth, and cause serious side effects in cancer patients, including nausea, vomiting, anorexia, hair loss, fatigue, bleeding, anemia, leukopenia, etc. Such side effects can not only affect the life quality of patients but also cause a cachexia, infection or heart failure, thereby leading to a risk of death. Furthermore, the cancer cachexia is a comprehensive metabolic syndrome, which is associated with the decrement of caloric uptake, the increment of static energy consumption, and the abnormal metabolism of protein, fat and carbohydrate. Cancer cachexia is characterized by weight loss, weakness, anorexia, fatigue, etc., and the continuous weight loss of patients cannot be avoided even if the food intake or nutrient uptake of the patient is enhanced.

There are researches showing that the immunosuppression of cancer cells is also associated with the development of cancer. Some cancer cells will bind to and induce immune cells via their surface antigens (e.g., programmed death-ligand 1) to trigger the immunosuppression of immune cells, such that the immune cells cannot be activated. The surface antigens of cancer cells such as the aforesaid programmed death-ligand 1 (PD-L1) are also called as “immune checkpoint antigens.” There are also researches showing that some anticancer medicaments (e.g., gemcitabine) will lead to an immunosuppression in tumor microenvironment (TME), thereby causing a drug resistance of cancer cells to immune system. Reference can be made to such as “Gemcitabine treatment promotes immunosuppressive microenvironment in pancreatic tumors by supporting the infiltration, growth, and polarization of macrophages”. Scientific reports. 2018 Aug. 10; 8(1):1-10, which is entirely incorporated hereinto by reference.

Therefore, the industry is still committed to studying medicaments and therapies for treating cancers. If one can effectively enhance the sensitivity of cancer cells to the anticancer medicament and/or decrease the administration amount of the anticancer medicament, the side effect(s) of the anticancer medicament can be reduced, thereby reducing the patient's burden and attenuating the symptom(s) of cachexia in patients. In addition, if the immunosuppression induced by the anticancer medicament can be reversed, the therapeutic effect(s) of the anticancer medicament will be further enhanced, and this will be beneficial to the cancer treatment.

### SUMMARY

Inventors of the present invention found that as compared to using an anticancer medicament alone, using a compound of formula (I) of the present invention or its salt(s) in combination with the anticancer medicament can enhance a sensitivity of cancer cells to the anticancer medicament and effectively decrease an administration amount of the anticancer medicament, thereby achieving the purposes of reducing a side effect of the anticancer medicament, reversing an immunosuppression induced by the anticancer medicament, and attenuating a symptom of cachexia in a subject with cancer.

Therefore, an objective of the present invention is to provide a use of an active ingredient in the manufacture of a pharmaceutical composition for use in combination with an anticancer medicament to enhance a sensitivity of cancer cells to the anticancer medicament, reduce a side effect of the anticancer medicament, reverse an immunosuppression induced by the anticancer medicament and/or attenuate a symptom of cachexia in a subject with cancer, wherein the active ingredient is selected from the group consisting of a compound of formula (I), a pharmaceutically acceptable salt of the compound of formula (I), and combinations thereof:

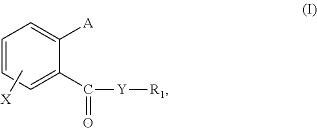

wherein A is a C1-C8 aliphatic hydrocarbyl optionally having a carbonyl; X is H or OH; Y is O; and R₁ is H or absent, with the proviso that when R₁ is absent, Y and A bond together to form a five-membered ring, and wherein the anticancer medicament is selected from the group consisting of a topoisomerase inhibitor, a microtubule assembly inhibitor, a platinum-based agent, an antimetabolite, and combinations thereof.

Another objective of the present invention is to provide a use of a first active ingredient and a second active ingredient in the manufacture of a pharmaceutical composition for treating a cancer, wherein the first active ingredient is selected from the group consisting of a compound of formula (I), a pharmaceutically acceptable salt of the compound of formula (I), and combinations thereof:

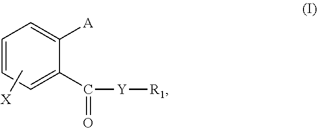

wherein A is a C1-C8 aliphatic hydrocarbyl optionally having a carbonyl; X is H or OH; Y is O; and R₁ is H or absent, with the proviso that when R₁ is absent, Y and A bond together to form a five-membered ring, and wherein the second active ingredient is selected from the group consisting of a topoisomerase inhibitor, a microtubule assembly inhibitor, a platinum-based agent, an antimetabolite, and combinations thereof.

Still another objective of the present invention is to provide a combination comprising a first component and a second component, wherein the first component is selected from the group consisting of a compound of formula (I), a pharmaceutically acceptable salt of the compound of formula (I), and combinations thereof:

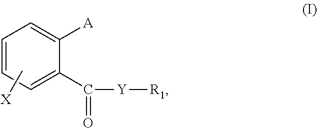

wherein A is a C1-C8 aliphatic hydrocarbyl optionally having a carbonyl; X is H or OH; Y is O; and R₁ is H or absent, with the proviso that when R₁ is absent, Y and A bond together to form a five-membered ring, and wherein the second component is selected from the group consisting of a topoisomerase inhibitor, a microtubule assembly inhibitor, a platinum-based agent, an antimetabolite, and combinations thereof. Preferably, the combination is in a form of a pharmaceutical composition or a kit. According to an embodiment of the combination of the present invention, the combination is for use in treating a cancer.

Still another objective of the present invention is to provide a method for treating a cancer, comprising administering to a subject in need the combination as mentioned above.

In the aforesaid use, combination or method of the present invention, regarding the compound of formula (I), A is preferably a C1-C6 aliphatic hydrocarbyl and R₁ is absent, more preferably, A is a C5 alkyl or alkenyl; or A is preferably a C1-C6 aliphatic hydrocarbyl having a carbonyl and R₁ is H, more preferably, A is a C5 alkyl or alkenyl having a carbonyl.

In the aforesaid use, combination or method of the present invention, if the pharmaceutical acceptable salt of the compound of formula (I) is involved, the pharmaceutical acceptable salt is preferably at least one of a lithium salt, a sodium salt, a potassium salt, a magnesium salt, a calcium salt, and a zinc salt.

In the aforesaid use, combination or method of the present invention, the anticancer medicament, second active ingredient, and second component is preferably independently selected from the group consisting of irinotecan, topotecan, etoposide, mitoxantrone, teniposide, azacitidine, 5-fluorouracil (5-FU), tegafur, TS-1, 6-mercaptopurine (6-MP), azathioprine, capecitabine, cladribine, clofarabine, cytosine arabinoside (Ara-C), decitabine, floxuridine, fludarabine, gemcitabine, hydroxyurea, methotrexate, nelarabine, pemetrexed, pentostatin, pralatrexate, thioguanine, trifluridine/tipiracil combination, cisplatin, oxaliplatin, paclitaxel, docetaxel, and combinations thereof.

In the aforesaid use, combination or method of the present invention, the cancer is preferably at least one of colorectal cancer, colon cancer, lung cancer, pancreatic cancer, bladder cancer, cholangiocarcinoma, rectal cancer, breast cancer, multiple myeloma, gynecologic tumor, brain cancer, testicular cancer, leukemia, lymphoma, pleural mesothelioma, gastric cancer, and liver cancer.

### BRIEF DESCRIPTION OF THE DRAWINGS

FIG. 1 shows an image of expressions of CD44ICD protein, PD-L1 protein, and GAPDH protein in Panc02 cells treated with (Z)-n-butylidenephthalide at different concentrations, wherein the expressions were analyzed by Western Blotting.

FIG. 2 shows images of pancreatic tumors in mice of each group (in which a portion circled by a dotted line represents a tumor), including the results of “Control” group, “LD” group (low dose), “HD” group (high dose), “Gem” group (gemcitabine), “LD+Gem” group, “TS-1” group, and “LD+TS-1” group, wherein the images were taken at the fifteenth day after orthotopically injecting Panc02 cells into the murine pancreas.

FIG. 3 shows a histogram of tumor sizes analyzed from the portions circled by dotted lines in FIG. 2 (* representing p value <0.05 as compared to the control group; ** representing p value <0.005 as compared to the control group).

FIG. 4 shows tumor sizes measured at the first, fifteenth and twenty-second days after orthotopically injecting Panc02 cells into the murine pancreas (shown by Photon Flux), including the results of “Cisplatin” group and “Z-BP+Cisplatin” group.

FIG. 5A to FIG. 5D show curve diagrams of survival rates of mice with pancreatic cancer for different treatments.

FIG. 6 shows an image of expressions of CD44 protein, CD44ICD protein, PD-1 protein, PD-L1 protein, p-Akt protein, Akt protein, and GAPDH protein in pancreatic tumor tissues of the mice with pancreatic cancer for different treatments.

### DESCRIPTION OF EMBODIMENTS

The detail techniques and some embodiments of the present invention will describe in the following, so that a person having original skills in the art can understand the features of the present invention. However, without departing from the spirit of the present invention, the present invention may be embodied in various embodiments and should not be limited to the embodiments described in the specification.

Unless otherwise indicated herein, the expressions “a,” “an,” “the,” or the like recited in the specification (especially in the claims) should include both the singular and plural forms. The term “subject” refers to human and non-human mammalians (e.g., dog, cat).

The definition of “response rate” refers to a ratio of patients who have a response to a treatment within any one period of an observation duration, and the response level (the degree of which the tumor size is decreased) can be divided into a complete response (tumor elimination), partial response (the tumor size is decreased by at least 50%). For example, an anticancer medicament with a “40% response rate” means that the medicament can provide an anticancer effect in 40% patient treated with the medicament, and the anticancer effect includes decreasing the tumor size by at least 50%, even eliminating the tumor completely.

There are clinic researches showing that some patients experience problems in a low response rate of medicament and a high cytotoxicity/tissue toxicity from chemotherapy. Inventors of the present invention found that as compared to using an anticancer medicament alone, using the compound of the present invention (i.e., a compound of formula (I)) or its salt(s) in combination with the anticancer medicament can enhance a sensitivity of cancer cells to the anticancer medicament and effectively decrease an administration amount of the anticancer medicament, thereby achieving the purposes of reducing a side effect of the anticancer medicament, reversing an immunosuppression induced by the anticancer medicament, and attenuating a symptom of cachexia in a subject with cancer.

Therefore, the present invention relates to a use of a compound of formula (I) and/or a pharmaceutically acceptable salt of the compound of formula (I) for treating a cancer:

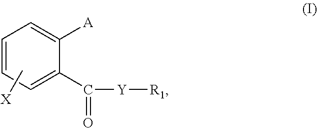

wherein A is a C1-C8 aliphatic hydrocarbyl optionally having a carbonyl; X is H or OH; Y is O; and R₁ is H or absent, with the proviso that when R₁ is absent, Y and A bond together to form a five-membered ring.

The use includes: (i) using the compound of formula (I) and/or the pharmaceutical acceptable salt of the compound of formula (I) in the manufacture of a pharmaceutical composition for use in combination with an anticancer medicament to enhance a sensitivity of cancer cells to the anticancer medicament, reduce a side effect of the anticancer medicament, reverse an immunosuppression induced by the anticancer medicament and/or attenuate a symptom of cachexia in a subject with cancer; (ii) using the compound of formula (I) and/or the pharmaceutically acceptable salt of the compound of formula (I) as a first active ingredient and an anticancer medicament as a second active ingredient in the manufacture of a pharmaceutical composition for treating a cancer; (iii) a combination, comprising the compound of formula (I) and/or the pharmaceutically acceptable salt of the compound of formula (I) as a first active ingredient and an anticancer medicament as a second active ingredient; and (iv) a method for treating a cancer, comprising administering the aforesaid combination to a subject in need.

In the use of the present invention, regarding the compound of formula (I), A is preferably a C1-C6 aliphatic hydrocarbyl (more preferably, A is a C5 alkyl or alkenyl), and R₁ is absent; or A is preferably a C1-C6 aliphatic hydrocarbyl having a carbonyl (more preferably, A is a C5 alkyl or alkenyl having a carbonyl), and R₁ is H. For example, in some embodiments of the use of the present invention, the compound of formula (I) is (Z)-n-butylidenephthalide, (E)-n-butylidenephthalide, 2-pentanolybenzoic acid or butylphthalide.

In the use of the present invention, examples of the pharmaceutically acceptable salt of the compound of formula (I) include a lithium salt, a sodium salt, a potassium salt, a magnesium salt, a calcium salt, and a zinc salt. In some embodiments of the use of the present invention, the pharmaceutically acceptable salt of the compound of formula (I) is a sodium salt such as sodium 2-pentanoylbenzoate.

In the use of the present invention, the compound of formula (I) and the pharmaceutically acceptable salt of the compound of formula (I) can be commercially available or be prepared by a synthesis method known in the field of the present invention.

Examples of the anticancer medicament suitable in the use of the present invention include a topoisomerase inhibitor, a microtubule assembly inhibitor, a platinum-based agent, an antimetabolite, and combinations thereof. Preferably, the anticancer medicament is selected from the group consisting of irinotecan, topotecan, etoposide, mitoxantrone, teniposide, azacitidine, 5-fluorouracil (5-FU), tegafur, TS-1 (i.e., a complex medicament containing a prodrug of 5-FU, tegafur), 6-mercaptopurine (6-MP), azathioprine (i.e., a prodrug of 6-MP), capecitabine, cladribine, clofarabine, cytosine arabinoside, decitabine, floxuridine, fludarabine, gemcitabine, hydroxyurea, methotrexate, nelarabine, pemetrexed, pentostatin, pralatrexate, thioguanine, trifluridine/tipiracil combination, cisplatin, oxaliplatin, paclitaxel, docetaxel, and combinations thereof. In some embodiments of the use of the present invention, the anticancer medicament is one or more of irinotecan, 5-fluorouracil, TS-1, gemcitabine, cisplatin, oxaliplatin, and paclitaxel.

The combination provided in accordance with the present invention can be a pharmaceutical composition or a kit. In an embodiment of the combination in accordance with the present invention, the combination is used for a cancer treatment. When the combination in accordance with the present invention is a kit, (1) the compound of formula (I) and/or the pharmaceutical acceptable salt as a first component and (2) the anticancer medicament as a second component are packaged separately and stored independently in different containers (e.g., a plastic bag, a plastic bottle, a glass bottle, an ampoule), and can be transported or sold either alone or in combination as a set. In addition, the kit can further comprise an instruction manual, which provides the procedures and program for the user to mix the components on-site for further processing and application.

In the use of the present invention, examples of the cancer include colorectal cancer, colon cancer, lung cancer (e.g., non-small-cell lung cancer), pancreatic cancer, bladder cancer, cholangiocarcinoma, rectal cancer, breast cancer, multiple myeloma, gynecologic tumor (e.g. cervical cancer, ovarian cancer, uterine cancer, vulvar cancer), brain cancer (e.g., glioblastoma), testicular cancer, leukemia (e.g., acute myeloid leukemia), lymphoma, pleural mesothelioma, gastric cancer, and liver cancer.

The pharmaceutical composition or the component(s) of the kit provided in accordance with the present invention can be administered systemically or topically, and can be delivered by various drug delivery system (DDS), wherein the suitable drug delivery system includes oral drug delivery system, transdermal drug delivery system, injectable drug delivery system, inhalation drug delivery system, and transmucosal drug delivery system, etc. For example, to enhance bioavailability, control drug release speed, target the lesion precisely and reduce side effects, the pharmaceutical composition or the component(s) of the kit provided in accordance with the present invention can be delivered by a liposome, a microcapsule, nanoparticles, or microneedles, but is not limited thereby.

Depending on the desired purpose(s), the pharmaceutical composition or the component(s) of the kit in accordance with the present invention can be provided in any suitable form without particular limitations. For example, the pharmaceutical composition or the component(s) of the kit can be administered to a subject in need by oral administration, transdermal administration (such as patch, ointment, etc.), corticospinal tract injection, intrathecal injection, intracerebral injection, intravenous injection (including drip infusion and bolus injection), intramuscular injection, subcutaneous injection, intraarterial injection, intraperitoneal injection, subcutaneous implantation, interstitial implantation, transrespiratory tract (e.g., spray, nasal drops, etc.), transmucosal (e.g., mouth-dissolving tablet, etc.), but is not limited thereby. Depending on the form and purpose(s), a suitable carrier can be chosen and used to provide the pharmaceutical composition or the component(s) of the kit, wherein the carrier is known and adopted in the art, including an excipient, a diluent, an auxiliary, a stabilizer, an absorption enhancer, a disintegrating agent, a hydrotropic agent, an emulsifier, an antioxidant, an adhesive, a binder, a tackifier, a dispersant, a suspending agent, a lubricant, a hygroscopic agent, etc.

As a dosage form for oral administration, the pharmaceutical composition or the component(s) of the kit can be provided in a form suitable for oral administration, wherein a liquid form suitable for oral administration includes a syrup, an oral solution, a suspension, an elixir, etc., and a solid form suitable for oral administration includes a powder, a granule, a troche, a dragee, an enteric-coated tablet, a chewable tablet, an effervescent tablet, a film-coated tablet, a capsule, a long-acting slow-release tablet, etc. The pharmaceutical composition or the component(s) of the kit provided in accordance with the present invention can comprise any pharmaceutically acceptable carrier that will not adversely affect the desired effects of at least one of the anticancer medicament and the compound of formula (I) and the pharmaceutical acceptable salt of the compound of formula (I). For example, the pharmaceutically acceptable carrier of the aforesaid liquid form includes, but is not limited to, water, saline, dextrose, glycerol, ethanol or its analogs, oil (e.g., olive oil, castor oil, cottonseed oil, peanut oil, corn oil, and germ oil), glycerin, polyethylene glycol, and combinations thereof; and the pharmaceutically acceptable carrier of the aforesaid solid form includes cellulose, starch, kaolinite, bentonite, sodium citrate, gelatin, agar, carboxymethyl cellulose, gum arabic, seaweed gel, glyceryl monostearate, calcium stearate, and combinations thereof.

As a dosage form for transdermal administration, the pharmaceutical composition or the component(s) of the kit of the present invention can also comprise a pharmaceutically acceptable carrier that will not adversely affect the desired effect(s) of at least one of the anticancer medicament and the compound of formula (I) and the pharmaceutical acceptable salt of the compound of formula (I), such as water, mineral oil, propylene glycol, polyethylene oxide, liquid petrolatum, sorbitan monosterate, and polysorbate 60. The pharmaceutical composition or the component(s) of the kit can be provided by any suitable method in a form suitable for transdermal administration, such as in the form of an emulsion, a cream, an oil, a gel (such as a hydrogel), a paste (such as a dispersing paste, an ointment), a lotion, a spray, and a patch (such as a microneedle patch), but is not limited thereby.

As for the form suitable for injections or drips, the pharmaceutical composition or the component(s) of the kit can comprise one or more ingredients, such as an isotonic solution, a salt-buffered saline (e.g., phosphate-buffered saline or citrate-buffered saline), a hydrotropic agent, an emulsifier, a 5% sugar solution, and other carriers to provide the pharmaceutical composition or the component(s) of the kit as an intravenous infusion, an emulsified intravenous infusion, a powder for injection, a suspension for injection, or a powder suspension for injection, etc. Alternatively, the pharmaceutical composition or the component(s) of the kit can be prepared as a pre-injection solid, and the desired injection is provided by dissolving the pre-injection solid in another solution or suspension or emulsifying it prior to being administered to a subject in need.

As a dosage form suitable for subcutaneous implantation or interstitial implantation, the pharmaceutical composition or the component(s) of the kit provided by the present invention can further comprise one or more ingredients, such a s an excipient, a stabilizer, a buffer, other carriers, etc., to be prepared in form as such as a wafer, a tablet, a pill, a capsule, etc., such that the pharmaceutical composition or the component(s) of the kit can be implanted into a subject and at least one of the anticancer medicament and the compound of formula (I) and the pharmaceutical acceptable salt of the compound of formula (I) contained therein can be slowly and continuously released to the surrounding tissue to achieve the effect of killing cancer cells at a locally stable high dose. For example, the pharmaceutical composition or the component(s) of the kit provided by the present invention can comprise a biocompatible polymer to prepare the pharmaceutical composition or the component(s) of the kit in the form of a wafer for subcutaneous implantation or interstitial implantation, but is not limited thereby. The biocompatible polymer can be commercially available or be prepared by a synthesis method known in the field of the present invention. For example, the biocompatible polymer can be a polyanhydride, such as p(CPP-SA) copolymer prepared from bis (p-carboxylphenoxy) propane and sebacic acid.

Regarding the pharmaceutical composition or the component(s) of the kit for transrespiratory tract administration, the pharmaceutical composition or the component(s) of the kit can be optionally aerosolized by any suitable approach to facilitate the entry of the pharmaceutical composition or the component(s) of the kit into the respiratory tract. For example, the pharmaceutical composition or the component(s) of the kit can be administered through a nebulizer or a pressurized container (e.g., nasal spray), but is not limited thereby. Alternatively, the pharmaceutical composition or the component(s) of the kit can be prepared as a nasal drop.

As for the pharmaceutical composition or the component(s) of the kit for transmucosal administration, the pharmaceutical composition or the component(s) of the kit provided by the present invention can comprise one or more ingredients, such as a penetrant, a surfactant, a viscosity modifier, a pH adjuster, a preservative, a stabilizer, an osmotic pressure regulator, and other carriers, to provide the pharmaceutical composition or the component(s) of the kit in the form of an eye drop, an eye ointment, a mouth-dissolving tablet, a suppository, a nasal spray, a nasal drop, etc.

Optionally, the pharmaceutical composition or the component(s) of the kit provided in accordance with the present invention can further comprise a suitable amount of an additive, such as a toner and/or a coloring agent for enhancing the palatability and the visual perception of the pharmaceutical composition or the kit, and a buffer, a conservative, a preservative, an antibacterial agent, and/or an antifungal agent for improving the stability and storability of the pharmaceutical composition or the kit.

The pharmaceutical composition or the component(s) of the kit provided in accordance with the present invention can optionally comprise one or more other active ingredients, to further enhance the effect of the composition or the kit or to increase the application flexibility and adaptability of the preparation thus provided, as long as the other active ingredient(s) does not adversely affect the desired effect(s) of the anticancer medicament and/or the compound of formula (I) and the pharmaceutical acceptable salt of the compound of formula (I) contained in the pharmaceutical composition or the kit of the present invention.

The pharmaceutical composition provided in accordance with the present invention contains at least about 0.0001, 0.0002, 0.0003, 0.0004, 0.0005, 0.001, 0.0015, 0.002, 0.0025, 0.003, 0.0035, 0.004, 0.0045, 0.005, 0.0055, 0.006, 0.0065, 0.007, 0.0075, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 5, 10, 15, 20, 25, 30, 35, 40, 45, 50, 55, 60, 65, 70, 75, 80, 85, 90, 95 or 100 wt % of at least one of the compound of formula (I) and the pharmaceutical acceptable salt of compound of formula (I) based on the total weight of the composition, and useful ranges may be selected from any two of these values, for example, about 0.0001 wt % to about 90 wt %, about 0.001 wt % to about 25 wt %, about 0.01 wt % to about 10 wt %, about 0.01 wt % to about 5 wt %, about 0.05 wt % to about 1 wt %, and about 0.05 wt % to about 0.5 wt %.

Depending on the need, age, body weight and health conditions of the subject and the purpose of application, the pharmaceutical composition or the kit provided in accordance with the present invention can be taken at various frequencies, such as once a day, multiple times a day, or once every few days, etc. The amount of at least one of anticancer medicament and/or the compound of formula (I) and the pharmaceutical acceptable salt of the compound of formula (I) in the pharmaceutical composition or the kit provided in accordance with the present invention can be adjusted, for example, to the amount that it should be taken or external used daily, depending on the need in the practical application.

In the method provided in accordance with the present invention, the administration route, administration frequency and range of administration amount of the pharmaceutical composition or the kit are all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

In the following Preparation Examples, the used materials and equipment are described as below:

1. Human pancreatic cancer cell line: Mia-PaCa2 cell (purchased from Bioresource Collection and Research Center (website: https://www.bcrc.firdi.org.tw/wwwbcrc/index.do); BCRC 60319), PANC-1 cell (purchased from Bioresource Collection and Research Center; BCRC 60494), AsPC-1 cell (purchased from Bioresource Collection and Research Center; BCRC 60494).

2. Mouse pancreatic cancer cell line: Panc02 cell (provided by Everfront Biotech Inc.).

3. Lung cancer cell line: A549 cell (purchased from Bioresource Collection and Research Center; BCRC 60074).

4. Brain cancer cell line: DBTRG-05MG cell (purchased from Bioresource Collection and Research Center; BCRC 60380).

5. Colorectal cancer cell line: HT-29 cell (purchased from Bioresource Collection and Research Center; BCRC 67003).

6. Liver cancer cell line: HepG2 (purchased from Bioresource Collection and Research Center; BCRC 60177).

7. Cell medium of Mia-PaCa2 cell: DMEM-HG medium (Dulbecco's modified Eagle's medium-High Glucose) containing L-glutamine, sodium pyruvate (purchased from Thermo Fisher Scientific), 10% fetal calf serum (FCS; purchased from Gibco; product number: 1939760), 1% penicillin/streptomycin (P/S; purchased from Simply; product number: CC502-0100), and 2.3% horse serum (purchased from Gibco; product number: 16050122).

8. Cell medium of PANC-1 cell: DMEM-HG medium containing 10% fetal calf serum and 1% penicillin/streptomycin.

9. Cell medium of AsPC-1 cell: RPMI1640 medium (purchased from HyClone) containing 10% fetal calf serum, 1% penicillin/streptomycin, 10 mM HEPES (purchased from Biomedicals; product number: 194549), and 1 mM sodium pyruvate.

10. Cell medium of Panc02 cell: RPMI1640 medium containing 10% fetal calf serum and 1% penicillin/streptomycin.

11. Cell medium of A549 cell: DMEM medium containing 10% fetal calf serum.

12. Cell medium of DBTRG-05MG cell: RPMI1640 medium containing 10% fetal calf serum and 1 mM sodium pyruvate.

13. Cell medium of HT-29 cell: RPMI1640 medium containing 10% fetal calf serum.

14. Cell medium of HepG2: DMEM medium containing 10% fetal calf serum.

15. (Z)-n-butylidenephthalide (Z—BP): provided by Everfront Biotech Inc.; purity 99.8%.

16. (E)-n-butylidenephthalide (E-BP): provided by Everfront Biotech Inc.; purity 98.01%.

17. 2-pentanolybenzoic acid (BP—OH): provided by Everfront Biotech Inc.; purity 99.6%.

18. Sodium 2-pentanoylbenzoate (BPONa): provided by Everfront Biotech Inc.; purity 99.7%.

19. Butylphthalide: provided by Everfront Biotech Inc.; purity ≥97%.

20. Gemcitabine (GEM): purchased from APEXBio; product number: A8437.

21. 5-fluorouracil (5-FU): purchased from Sigma; product number: F6627.

22. Irinotecan (CPT-11): purchased from Sigma; product number: I1406.

23. Cisplatin (CDDP): purchased from Sigma; product number: C2210000.

24. Oxaliplatin (OXA): purchased from Sigma; product number: 61825-94-3.

25. Paclitaxel (PTX): purchased from Sigma; product number: 33069-62-4.

26. TS-1: provided by Everfront Biotech Inc.

27. MTT (Thiazolyl Blue tetrazolium bromide, 3-[4,5-dimethylthiahiazo-2-yl]-2,4-dipheny-tetrazolium bromide): purchased from ALFA Aesar™; product number: L11939-000000-16AF.

28. ELISA reader: purchased from Thermo Fisher Scientific; model number: 22662.

29. C57BL/6J mouse (body weight: 18 to 22 g): purchased from National Laboratory Animal Center, Taipei, Taiwan.

30. Antibodies for Western Blotting: anti-Akt antibody (purchased from Cell Signaling Technology; product number: #9272); anti-phospho-Akt (Ser473) antibody (purchased from Cell Signaling Technology; product number: #9271), anti-CD44 antibody (purchased from Abcam; product number: #ab24504), anti-PD-L1 antibody (purchased from Abcam; product number: #ab238697), anti-PD-1 antibody (purchased from BioLegend; product number: #367402); anti-GAPDH antibody (purchased from Genetex; product number: GTX100118).

### Example 1: Effects of the Compound of the Present Invention and Different Anticancer Medicaments on Killing Cancer Cells

In this example, the effects of the compound of the present invention and different anticancer medicaments on killing cancer cells were studied by MTT (Thiazolyl Blue tetrazolium bromide, 3-[4,5-dimethylthiahiazo-2-yl]-2,4-dipheny-tetrazolium bromide) cell viability analysis.

1-1. Effects of the Compound of the Present Invention and Different Anticancer Medicaments on Killing Pancreatic Cancer Cells

The pancreatic cancer cell lines, PANC-1 cells, Mia-PaCa2 cells, AsPC-1 cells, and Panc02 cells, were cultured in each well of a 96-well plate (1×10⁴ cells/well; the 96-well plate was plated in an incubator kept at 37° C. and 5% CO₂) for 24 hours. Thereafter, each of the aforesaid cell lines was cultured with cell medium containing gemcitabine, cisplatin, 5-fluorouracil, irinotecan, oxaliplatin, paclitaxel, (Z)-n-butylidenephthalide, (E)-n-butylidenephthalide, 2-pentanolybenzoic acid, sodium 2-pentanoylbenzoate, and butylphthalide, for 24 hours, 48 hours and 72 hours, respectively. Then, MTT was independently added into each well of the 96-well plate (final concentration of MTT in the cell medium of each well was 0.5 mg/ml), and the 96-well plate was plated in an incubator kept at 37° C. and 5% CO₂ for 1.5 hours. After removing the cell medium, 100 μL dimethyl sulfoxide was added and an absorbance thereof at 595 nm was measured by an ELISA reader, thereby calculating the cell viability and calculating the half maximal inhibitory concentrations (IC₅₀) of gemcitabine, cisplatin, 5-fluorouracil, irinotecan, oxaliplatin, paclitaxel, (Z)-n-butylidenephthalide, (E)-n-butylidenephthalide, 2-pentanolybenzoic acid, sodium 2-pentanoylbenzoate, and butylphthalide in each of pancreatic cancer cell lines. The results are shown in Table 1.

TABLE 1
IC₅₀ (unit: μg/ml)

| | PANC-1 | Mia-PaCa2 | AsPC-1 | Panc02 |
|---|---|---|---|---|
| gemcitabine | 1.53 ± 0.30 | 0.85 ± 0.36 | 6.56 ± 0.82 | 3.77 × 10⁻³ ± 0.63 × 10⁻⁴ |
| cisplatin | 35.87 ± 1.23 | 32.86 ± 1.08 | 21.56 ± 1.05 | 53.36 ± 1.74 |
| 5-fluorouracil | 8.45 ± 0.35 | 4.90 ± 0.27 | 25.36 ± 3.91 | 67.28 × 10⁻³ ± 2.41 × 10⁻³ |
| irinotecan | 38.45 ± 1.08 | 21.67 ± 0.82 | 54.40 ± 2.13 | 69.48 ± 1.01 |
| oxaliplatin | 18.79 ± 1.35 | 21.81 ± 1.60 | 42.60 ± 7.61 | 8.43 ± 0.83 |
| paclitaxel | 19.6 × 10⁻³ ± 1.16 × 10⁻³ | 4.58*10⁻³ ± 0.77*10⁻³ | 58.01*10⁻³ ± 2.78*10⁻³ | 12.44 × 10⁻³ ± 0.65 × 10⁻³ |
| (Z)-n-butylidenephthalide | 41.19 ± 2.81 | 55.89 ± 1.35 | 83.32 ± 2.56 | 32.88 ± 2.325 |
| (E)-n-butylidenephthalide | 778.6 ± 26.90 | 628.9 ± 16.46 | 131.1 ± 43.32 | 513.6 ± 9.21 |
| 2-pentanolybenzoic | 980.5 ± 22.64 | 693.5 ± 19.57 | 490.2 ± 19.69 | 769.9 ± 15.13 |
| acid | | | | |
| sodium | 687.0 ± 27.14 | 469.2 ± 18.56 | 624.3 ± 49.64 | 750.1 ± 28.68 |
| 2-pentanoylbenzoate | | | | |
| butylphthalide | >200 | 198.9 ± 7.69 | >300 | — |

1-2. Effects of the Compound of the Present Invention and 5-Fluorouracil on Killing Cancer Cells

The lung cancer cell line (A549 cell), liver cancer cell line (HepG2 cell), colorectal cancer cell line (HT-29 cell), and brain cancer cell line (DBTRG-05MG cell) were cultured in each well of a 96-well plate (1×10⁴ cell/well; the 96-well plate was plated in an incubator kept at 37° C. and 5% CO₂) for 24 hours. Thereafter, each of the aforesaid cell lines were cultured with cell medium containing 5-fluorouracil, (Z)-n-butylidenephthalide, and 2-pentanolybenzoic acid for 24 hours, 48 hours and 72 hours. Then, the MTT solution was independently added into each well of the 96-well plate (0.5 mg/ml), and the 96-well plate was plated in an incubator kept at 37° C. and 5% CO₂ for 1.5 hours. After removing the cell medium, 100 μL dimethyl sulfoxide was added and an absorbance thereof at 595 nm was measured by an ELISA reader, thereby calculating the cell viability and calculating the half maximal inhibitory concentrations (IC₅₀) of 5-fluorouracil, (Z)-n-butylidenephthalide, and 2-pentanolybenzoic acid in each of cancer cell lines. The results are shown in Table 2.

TABLE 2
IC₅₀ (unit: μg/ml)

| | A549 | HepG2 | HT-29 | DBTRG-05MG |
|---|---|---|---|---|
| 5-fluorouracil | 8.55 ± 0.22 | 4.44 ± 0.25 | 20.80 ± 0.91 | 55.45 ± 6.10 |
| (Z)-n-butylidenephthalide | 63.60 ± 0.06 | 81.10 ± 0.02 | 52.90 ± 0.01 | 132.6 ± 0.00 |
| 2-pentanolybenzoic acid | 795.5 ± 0.02 | 1141.5 ± 0.01 | 443.8 ± 0.04 | 392.8 ± 0.01 |

### Example 2: Effects of the Compound of the Present Invention in Combination with an Anticancer Medicament

2-1. (Z)-n-Butylidenephthalide and 5-Fluorouracil

The pancreatic cancer cell lines (PANC-1 cell, Mia-PaCa2 cell, and AsPC-1 cell), lung cancer cell line (A549 cell), liver cancer cell line (HepG2 cell), colorectal cancer cell line (HT-29 cell), and brain cancer cell line (DBTRG-05MG cell) were cultured in each well of a 96-well plate (1×10⁴ cell/well; the 96-well plate was plated in an incubator kept at 37° C. and 5% CO₂) for 24 hours. Thereafter, both (Z)-n-butylidenephthalide and 5-fluorouracil were added into the cell medium of each cell line simultaneously. After plating the 96-well plate in an incubator kept at 37° C. and 5% CO₂ for 1.5 hours, the cell medium was removed and 100 μL dimethyl sulfoxide was added. Then, an absorbance thereof at 595 nm was measured by an ELISA reader, thereby calculating the cell viability and calculating the half maximal inhibitory concentrations (IC₅₀) of each cancer cell line for the aforesaid processes. Finally, a combination index (CI) was calculated by the equation A. The results are shown in Table 3.

$$CI = \frac{D 1}{D \times 1} + \frac{D 2}{D \times 2} . \qquad Equation A$$

(D)1, (D)2 independently represent the IC₅₀ of using a medicament 1 in combination with a medicament 2, and (Dx)1, (Dx)2 independently represent the IC₅₀ of using the aforesaid two medicaments alone. If the combination index (CI) is less than 1, the use of combining two medicaments is considered to have a synergistic effect.

TABLE 3
CI values of using (Z)-n-butylidenephthalide in combination
with 5-fluorouracil for killing cancer cells

| Cell line | CI |
|---|---|
| PANC-1 | 0.39 |
| Mia-PaCa2 | 0.27 |
| AsPC-1 | 0.58 |
| A549 | 0.34 |
| HepG2 | 0.46 |
| HT-29 | 0.26 |
| DBTRG-05MG | 0.22 |

The results of Table 3 show that all the CI values of using (Z)-n-butylidenephthalide in combination with 5-fluorouracil for killing cancer cells are less than 1, and thus can provide a synergistic effect.

2-2. (Z)-n-Butylidenephthalide and Other Anticancer Medicaments

According to the method of Example 2-1, the pancreatic cancer cells were treated by (Z)-n-butylidenephthalide in combination with other anticancer medicaments, and the experimental data was calculated to obtain the CI values of the aforesaid combined uses for killing pancreatic cancer cells. The results are shown in Table 4.

TABLE 4
CI values of using (Z)-n-butylidenephthalide in combination with
other anticancer medicaments for killing pancreatic cancer cells

| | PANC-1 | Mia-PaCa2 | AsPC-1 | Panc02 |
|---|---|---|---|---|
| Z-BP + gemcitabine | 0.21 | 0.64 | 0.44 | 0.67 |
| Z-BP + cisplatin | 0.38 | 0.43 | 0.50 | 0.39 |
| Z-BP + irinotecan | 0.61 | 0.36 | 0.42 | 0.41 |
| Z-BP + oxaliplatin | 0.57 | 0.90 | 0.80 | 0.87 |
| Z-BP + paclitaxel | 0.19 | 0.54 | 0.49 | 0.95 |

The results of Table 4 show that all the CI values of using (Z)-n-butylidenephthalide in combination with other anticancer medicaments for killing cancer cells are less than 1, and thus can provide a synergistic effect.

2-3. (E)-n-Butylidenephthalide and Anticancer Medicaments

According to the method of Example 2-1, the pancreatic cancer cells were treated by (E)-n-butylidenephthalide in combination with anticancer medicaments such as oxaliplatin, paclitaxel, gemcitabine, 5-fluorouracil, and the experimental data was calculated to obtain the CI values of the aforesaid combined uses for killing pancreatic cancer cells. The results are shown in Table 5 to Table 7.

TABLE 5
CI values of using (E)-n-butylidenephthalide (E-BP) in combination
with oxaliplatin or paclitaxel for killing pancreatic cancer cells

| | PANC-1 | Mia-PaCa2 | Panc02 |
|---|---|---|---|
| E-BP + oxaliplatin | 0.89 | 0.96 | 0.54 |
| E-BP + paclitaxel | 0.69 | 0.97 | 0.73 |

TABLE 6
CI value of using (E)-n-butylidenephthalide (E-BP) in combination
with gemcitabine for killing pancreatic cancer cells

| | PANC-1 | Mia-PaCa2 |
|---|---|---|
| E-BP + gemcitabine | 0.95 | 0.64 |

TABLE 7
CI value of using (E)-n-butylidenephthalide (E-BP) in combination
with 5-fluorouracil for killing pancreatic cancer cells

| | Mia-PaCa2 |
|---|---|
| E-BP + 5-fluorouracil | 0.79 |

The results of Table 5 to Table 7 show that all the CI values of using (E)-n-butylidenephthalide in combination with the anticancer medicaments for killing cancer cells are less than 1, and thus can provide a synergistic effect.

2-4. 2-Pentanolybenzoic Acid (BP—OH) and Anticancer Medicaments

According to the method of Example 2-1, the pancreatic cancer cells (PANC-1, Mia-PaCa2, AsPC-1, and Panc02), lung cancer cell line (A549 cell), liver cancer cell line (HepG2 cell), colorectal cancer cell line (HT-29 cell), and brain cancer cell line (DBTRG-05MG cell) were treated by 2-pentanolybenzoic acid (BP—OH) in combination with anticancer medicaments such as 5-fluorouracil, gemcitabine, oxaliplatin, paclitaxel, and the experimental data was calculated to obtain the CI values of the aforesaid combined uses for killing each cancer cells. The results are shown in Table 8 to Table 11.

TABLE 8
CI values of using 2-pentanolybenzoic acid (BP-OH)
in combination with 5-fluorouracil for killing cancer cells

| Cell line | CI |
|---|---|
| PANC-1 | 0.57 |
| Mia-PaCa2 | 0.84 |
| AsPC-1 | 0.96 |
| Panc02 | 0.88 |
| A549 | 0.54 |
| HepG2 | 0.73 |
| HT-29 | 0.79 |
| DBTRG-05MG | 0.80 |

TABLE 9
CI values of using 2-pentanolybenzoic acid (BP-OH) in combination
with gemcitabine for killing pancreatic cancer cells

| | PANC-1 | Mia-PaCa2 |
|---|---|---|
| BP-OH + gemcitabine | 0.93 | 0.49 |

TABLE 10
CI values of using 2-pentanolybenzoic acid (BP-OH) in combination
with oxaliplatin for killing pancreatic cancer cells

| | PANC-1 | Mia-PaCa2 | Panc02 |
|---|---|---|---|
| BP-OH + oxaliplatin | 0.92 | 0.79 | 0.23 |

TABLE 11
CI values of using 2-pentanolybenzoic acid (BP-OH) in combination
with paclitaxel for killing pancreatic cancer cells

| | PANC-1 | Panc02 |
|---|---|---|
| BP-OH + paclitaxel | 0.61 | 0.52 |

The results of Table 8 to Table 11 show that all the CI values of using 2-pentanolybenzoic acid (BP—OH) in combination with the anticancer medicaments for killing cancer cells are less than 1, and thus there is a synergistic effect.

2-4. Sodium 2-Pentanoylbenzoate (BPONa) and Anticancer Medicaments

According to the method of Example 2-1, the pancreatic cancer cells were treated by sodium 2-pentanoylbenzoate (BPONa) in combination with anticancer medicaments such as oxaliplatin, gemcitabine, 5-fluorouracil, paclitaxel, and the experimental data was calculated to obtain the CI values of the aforesaid combined uses for killing pancreatic cancer cells. The results are shown in Table 12 to Table 14.

TABLE 12
CI values of using sodium 2-pentanoylbenzoate (BPONa)
in combination with oxaliplatin for killing pancreatic cancer cells

| | PANC-1 | Mia-PaCa2 | AsPC-1 | Panc02 |
|---|---|---|---|---|
| BPONa + oxaliplatin | 0.70 | 0.93 | 0.89 | 0.22 |

TABLE 13
CI values of using sodium 2-pentanoylbenzoate (BPONa)
in combination with gemcitabine for killing pancreatic cancer cells

| | Mia-PaCa2 | AsPC-1 | Panc02 |
|---|---|---|---|
| BPONa + gemcitabine | 0.82 | 0.93 | 0.55 |

TABLE 14
CI values of using sodium 2-pentanoylbenzoate (BPONa)
in combination with gemcitabine for killing pancreatic cancer cells

| | PANC-1 | Panc02 |
|---|---|---|
| BPONa + 5-fluorouracil | 0.66 | 0.78 |
| BPONa + paclitaxel | 0.57 | 0.60 |

The results of Table 12 to Table 14 show that all the CI values of using sodium 2-pentanoylbenzoate (BPONa) in combination with the anticancer medicaments for killing cancer cells are less than 1, and thus can provide a synergistic effect.

It can be seen from the results of this example, as compared to using an anticancer medicament alone, using the compound of formula (I) of the present invention or its salt(s) in combination with the anticancer medicament can enhance a sensitivity of cancer cells to the anticancer medicament and effectively decrease an administration amount of the anticancer medicament, thereby achieving the purposes of reducing a side effect of the anticancer medicament, reversing an immunosuppression induced by the anticancer medicament, and attenuating a symptom of cachexia in a subject with cancer.

### Example 3: Effects of the compound of the present invention on decreasing the expressions of CD44 and PD-L1

It is known that the programmed death-ligand 1 (PD-L1) on the surface of cancer cells can bind to the programmed death 1 (PD-1) on the surface of immune cells, thereby causing the death of immune cells. Furthermore, CD44 and CD44ICD can enhance the expression of PD-L1, and the down-regulation of CD44 can lead to an inhibition of cancer cell growth. Reference can be made to such as CD44 promotes PD-L1 expression and its tumor-intrinsic function in breast and lung cancers. Cancer Research. 2020 Feb. 1; 80(3):444-457, which is entirely incorporated hereinto by reference.

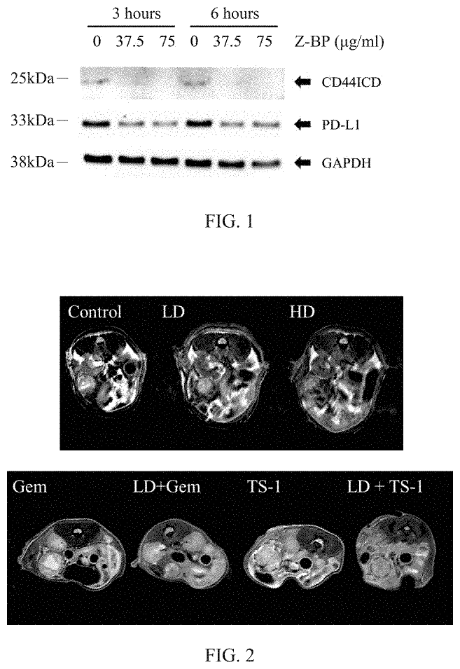

The Panc02 cells (pancreatic cancer cell line) were cultured with 37.5 and 75 μg/ml of (Z)-n-butylidenephthalide for 6 hours (a portion of cells were harvested after culturing for 3 hours), respectively. Thereafter, proteins of the cells were extracted, and the expressions of CD44 intracellular domain (CD44ICD) protein and PD-L1 protein in the cancer cells treated with (Z)-n-butylidenephthalide were measured by Western Blotting. Furthermore, the expression of GAPDH protein was measured as an internal control. The results are shown in FIG. 1.

It can be seen from FIG. 1 that the expressions of CD44ICD protein and PD-L1 protein in the Panc02 cells were both decreased as the concentration of (Z)-n-butylidenephthalide was increased, wherein the CD44ICD protein was completely inhibited at 6 hours. The aforesaid results show that in addition to inhibiting the growth of cancer cells, (Z)-n-butylidenephthalide can further be effective in inhibiting the CD44ICD protein of cancer cells, thereby achieving the effects on inhibiting the expressions of immune checkpoint antigens. Accordingly, the binding of cancer cells and immune cells can be blocked and thus reverses the immunosuppression induced by an anticancer medicament.

### Example 4: Use of the Compound of the Present Invention in Combination with an Anticancer Medicament for Treating Cancer

4-1. Establishment of Animal Model

According to the requirements of Institutional Animal Care and Use Committee (IACUC) of Dong Hwa University, C57BL/6J mice were feed in the experimental animal center of Dong Hwa University until 8 to 10 weeks old. Then, the stably constructed Panc02 cells transfected by Luc-eGFP were in-situ injected into the mice pancreases (1×10⁶ cells/0.02 ml/mouse). Thereafter, the tumor sizes of mice pancreases were analyzed through the animal imaging results, and the mice were equally divided into nine group depending on their tumor sizes and treated by the following conditions for three to four weeks:

(1) “Control (Ctl.)” group (5 mice): untreated, only orally administering vehicle (containing none of the compound of the present invention and other anticancer medicaments) every day.

(2) “LD” group (5 mice): orally administering a low dose (12.5 mg/kg body weight) of (Z)-n-butylidenephthalide every day.

(3) “HD” group (5 mice): orally administering a high dose (25 mg/kg body weight) of (Z)-n-butylidenephthalide every day.

(4) “Gem” group (5 mice): intraperitoneally injecting 100 mg/kg body weight of gemcitabine (GEM) per three days.

(5) “LD+Gem” group (5 mice): orally administering 12.5 mg/kg body weight of (Z)-n-butylidenephthalide every day, and intraperitoneally injecting 50 mg/kg body weight of gemcitabine (GEM) per three days.

(6) “TS-1” group (5 mice): after orally administering 100 mg/kg body weight of TS-1 every day for five continuous days, stopping the oral administration of TS-1 for two days.

(7) “LD+TS-1” group (2 mice): orally administering 12.5 mg/kg body weight of (Z)-n-butylidenephthalide every day, and after orally administering 50 mg/kg body weight of TS-1 every day for five continuous days, stopping the oral administration of TS-1 for two days.

(8) “Cisplatin” group (3 mice): intraperitoneally injecting 2.5 mg/kg body weight of cisplatin per seven days.

(9) “Z-BP+Cisplatin” group (3 mice): orally administering 6.25 mg/kg body weight of (Z)-n-butylidenephthalide every day, and intraperitoneally injecting 1.25 mg/kg body weight of cisplatin per seven days.

4-2. Observation of Tumor Size (T2-Weighted Magnetic Resonance Imaging)

The stably constructed Panc02 cells transfected by Luc-eGFP were in-situ injected into the mice of each group of Example 4-1, and after determining the growth of in-situ pancreatic tumor, the medicament treatments were conducted for 14 days (i.e., the fifteenth day). In this duration, the pancreatic tumors of mice in each group were analyzed and observed by T₂-weighted MRI and taken pictures for records. The results are shown in FIG. 2. The tumor (a portion circled by a dotted line) sizes of mice of each group in FIG. 2 were analyzed by amide software. The results are shown in FIG. 3.

Gemcitabine (GEM) and TS-1 are medicaments for treating a cancer in clinical. However, as shown in FIG. 3, the tumor sizes of mice in “Gem” group and “TS-1” group are bigger than that of untreated “Control” group (also called as “Ctl.” Group). It can be seen from the aforesaid results that gemcitabine (GEM) and TS-1 will induce an immunosuppression in tumor microenvironment (TME), thereby causing a drug resistance of cancer cells for immune system.

FIG. 3 also shows that the tumor sizes of “LD+Gem” group are significantly smaller than that of “Gem” group, and the tumor sizes of “LD+TS-1” group are significantly smaller than that of “TS-1” group. It can be seen from the aforesaid results that using the compound of the present invention or its salt(s) in combination with an anticancer medicament can effectively reverse an immunosuppression induced by the anticancer medicament and much effectively inhibit the tumor growth.

4-3. Observation of Tumor Size (IVIS Imaging System)

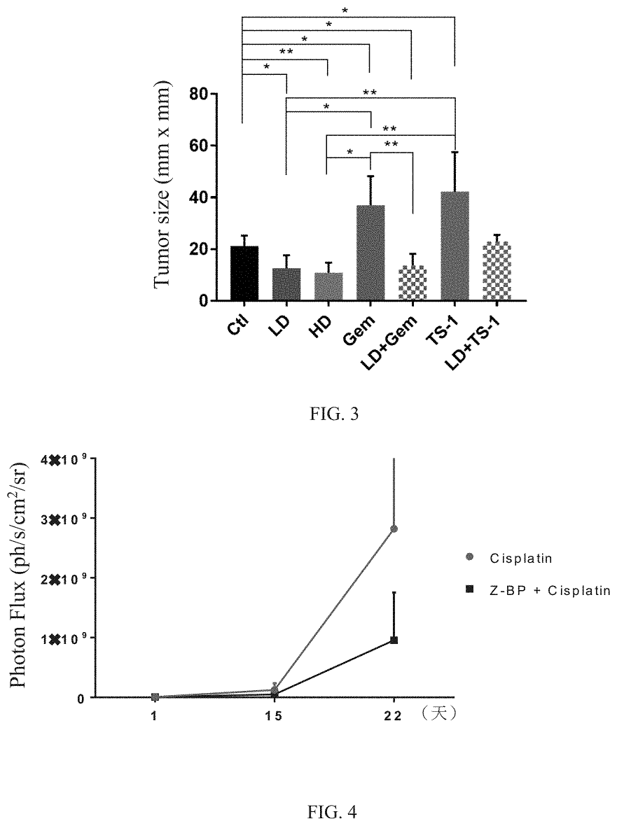

At the first, fifteenth and twenty-second days after in-situ injecting the stably constructed Panc02 cells transfected by Luc-eGFP into the mice of each group of Example 4-1, the tumor sizes of mice in “Cisplatin” group and “Z-BP+Cisplatin” group were detected by the IVIS imaging system (through measuring Photon Flux; unit: ph/s/cm²/sr). The results are shown in FIG. 4.

It can be seen from FIG. 4 that as compared to the “Cisplatin” group, the photon flux obtained from the “Z-BP+Cisplatin” group at the 22th day is significantly lower. In other words, the tumor sizes of “Z-BP+Cisplatin” group is significantly less than that of “Cisplatin” group. The aforesaid results also show that using the compound of the present invention or its salt(s) in combination with an anticancer medicament can effectively enhance the effect of anticancer medicament on inhibiting the tumor growth.

4-4. Observation of Survival

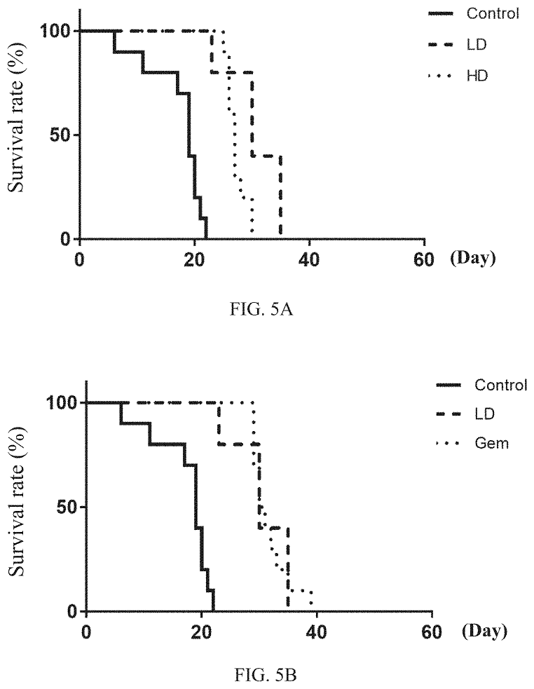

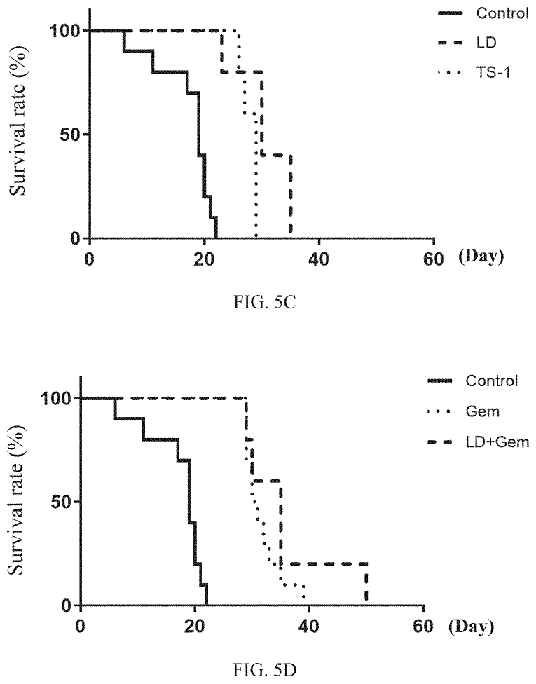

According to the method of Example 4-1, the pancreatic cancer mice were established and the survivals of mice in each group were observed and recorded every day. The results are shown in Table 15 and FIG. 5A to FIG. 5D.

TABLE 15

| | | Median of | Average of | Fold (as | | P value (as |
|---|---|---|---|---|---|---|
| | | overall | overall | compared | P value (as | compared to |
| | Number | survival | survival | to Ctl. | compared to | TS-1 group or |
| Group | of mice | (day) | (day) | group) | Ctl. group) | Gem group) |
| Ctl. | 10 | 19 | 17.4 ± 1.586 | — | — | — |
| LD | 5 | 30 | 30.6 ± 2.205 | 1.8 | 0.0003 | 0.0129 |
| | | | | | | (as compared to |
| | | | | | | TS-1 group) |
| HD | 10 | 27 | 27.2 ± 0.533 | 1.6 | 0.0001 | — |
| Gem | 10 | 30 | 31.7 ± 1.023 | 1.8 | 0.0001 | — |
| LD + Gem | 5 | 35 | 35.8 ± 3.76 | 2.1 | 0.0001 | 0.0215 |
| | | | | | | (as compared to |
| | | | | | | Gem group) |
| TS-1 | 5 | 29 | 28 ± 0.6325 | 1.6 | 0.0005 | — |

It can be seen from Table 15 and FIG. 5A to FIG. 5D that the survival days of mice in “LD” group are more than that of “Control” group (also called as “Ctl” group) and “TS-1” group. In addition, the survival days of mice in the “LD” group and “Gem” group are 1.8-fold of that of the “Control” group, and the survival days of mice in the “LD+Gem” group are 2.1-fold of that of the “Control” group.

4-5. Observation of Protein Expression

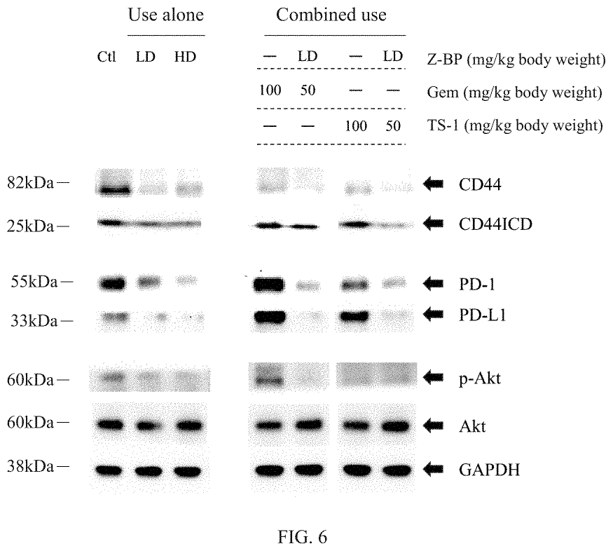

After accomplishing the observations of Examples 4-2 to 4-3, the mice in each group were sacrificed and the tissues of pancreatic tumor thereof were collected to extracting proteins. Then, the expressions of CD44 protein, CD44ICD protein, PD-1 protein, PD-L1 protein, p-Akt protein, and Akt protein in the protein sample of each group were detected by Western Blotting. Furthermore, the expression of GAPDH protein serves as an internal control. The results are shown in FIG. 6.

It can be seen from FIG. 6 that the expressions of PD-1 protein and PD-L1 protein in the mice of the “Gem” group and “TS-1” group are more than that of the untreated “Control” group (also called as “Ctl” group). The aforesaid results also show that gemcitabine (GEM) and TS-1 will induce an immunosuppression in tumor microenvironment (TME), thereby causing a drug resistance of cancer cells for immune system.

FIG. 6 also shows that the expressions of CD44 protein, CD44ICD protein, PD-1 protein and PD-L1 protein in the “LD+Gem” group are significantly less than that of the “Gem” group, and the expressions of CD44 protein, CD44ICD protein, PD-1 protein and PD-L1 protein in the “LD+TS-1” group are significantly less than that of the “TS-1” group. The aforesaid results show that (Z)-n-butylidenephthalide is effective in inhibiting the expressions of CD44 protein and CD44ICD protein in cancer cell, and further inhibiting the expressions of immune checkpoint antigens such as PD-1 protein, PD-L1 protein. Accordingly, (Z)-n-butylidenephthalide can block the binding of cancer cells and immune cells, thereby reversing an immunosuppression induced by an anticancer medicament. The aforesaid results also show that using the compound of the present invention or its salt(s) in combination with an anticancer medicament can effectively reverse an immunosuppression induced by the anticancer medicament, and thus can be beneficial to enhance the therapeutic effect(s) of anticancer medicament.

In addition, it can also be seen from FIG. 6, the expression of p-Akt protein (i.e., activated Akt protein) is decreased depending on the down-regulation of PD-L1 protein. The aforesaid results can evidence that PD-L1 signaling pathway was inhibited.

As seen in the animal test of this example, using the compound of the present invention or its salt(s) in combination with an anticancer medicament can enhance a sensitivity of cancer cell to the anticancer medicament in animal body, and effectively reduce the administration amount of anticancer medicament, thereby achieving the purposes of reducing a side effect of the anticancer medicament, reversing an immunosuppression induced by the anticancer medicament, and attenuating a symptom of cachexia in a subject with cancer.

1. (canceled)

2. (canceled)

3. (canceled)

4. (canceled)

5. (canceled)

6. (canceled)

7. (canceled)

8. (canceled)

9. (canceled)

10. (canceled)

11. A combination comprising a first component and a second component, wherein the first component is selected from the group consisting of a compound of formula (I), a pharmaceutically acceptable salt of the compound of formula (I), and combinations thereof: 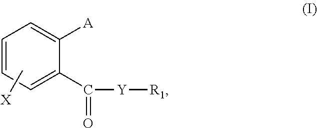
wherein,
A is a C1-C8 aliphatic hydrocarbyl optionally having a carbonyl;
X is H or OH;
Y is O; and
R₁ is H or absent, with the proviso that:
(i) when R₁ is H, the second component is selected from the group consisting of a topoisomerase inhibitor, a microtubule assembly inhibitor, a platinum-based agent, an antimetabolite, and combinations thereof; and
(ii) when R₁ is absent, Y and A bind together to form a five-membered ring, and the second component is selected from the group consisting of 5 fluorouracil (5-FU), irinotecan, oxaliplatin, gemcitabine, and combinations thereof.

12. The combination of claim 11, wherein A is a C1-C6 aliphatic hydrocarbyl, and R₁ is absent.

13. The combination of claim 11, wherein A is a C1-C6 aliphatic hydrocarbyl having a carbonyl, and R₁ is H.

14. The combination of claim 12, wherein A is a C5 alkyl or alkenyl.

15. The combination of claim 13, wherein A is a C5 alkyl or alkenyl having a carbonyl.

16. The combination of claim 13, wherein the pharmaceutically acceptable salt is at least one of a lithium salt, a sodium salt, a potassium salt, a magnesium salt, a calcium salt, and a zinc salt.

17. The combination of claim 11, wherein the second component is selected from the group consisting of irinotecan, topotecan, etoposide, mitoxantrone, teniposide, azacitidine, 5-fluorouracil, tegafur, TS-1, 6-mercaptopurine, azathioprine, capecitabine, cladribine, clofarabine, cytosine arabinoside, decitabine, floxuridine, fludarabine, gemcitabine, hydroxyurea, methotrexate, nelarabine, pemetrexed, pentostatin, pralatrexate, thioguanine, trifluridine/tipiracil combination, cisplatin, oxaliplatin, paclitaxel, docetaxel, and combinations thereof, when R₁ is H.

18. The combination of claim 11, wherein the combination is in a form of a pharmaceutical composition or a kit.

19. The combination of claim 11, which is used for treating cancer.

20. A method for treating a cancer, comprising administering to a subject in need the combination of claim 11.

21. The method of claim 20, wherein the cancer is at least one of colorectal cancer, colon cancer, lung cancer, pancreatic cancer, bladder cancer, cholangiocarcinoma, rectal cancer, breast cancer, multiple myeloma, gynecologic tumor, brain cancer, testicular cancer, leukemia, lymphoma, pleural mesothelioma, gastric cancer, and liver cancer.

22. The method of claim 20, wherein the cancer is at least one of colorectal cancer, colon cancer, lung cancer, pancreatic cancer, rectal cancer, brain cancer, and liver cancer.

23. The method of claim 20, which is for attenuating a symptom of cachexia in the subject.
